(19) United States
(12) Patent Application Publication
(10) Pub. No.: US 2009/0112155 A1
(43) Pub. Date: Apr. 30, 2009

(54) Micro Diaphragm Pump

(75) Inventors: Mitch Zhao, San Jose, CA (US); Peter Krulevitch, Pleasanton, CA (US); David Knight, Mountain View, CA (US); Anthony Lam, Fremont, CA (US); Sean O'Connor, West Chester, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan, Inc., Milpitas, CA (US)

(21) Appl. No.: 12/261,426

(22) Filed: Oct. 30, 2008

Related U.S. Application Data
(60) Provisional application No. 60/983,827, filed on Oct. 30, 2007.

(51) Int. Cl. A61M 5/152; F04B 49/00

(52) U.S. Cl. 604/67; 417/63; 604/153

(57) ABSTRACT

The invention relates to micropumps for infusing fluids. More specifically, the present disclosure describes and illustrates a micropump design that may be useful for infusing insulin into a diabetic patient. The disclosed design employs a pump chamber that has a diaphragm and a plurality of check valves that are configured to avoid leakage from the reservoir through the pump engine and into an infusion device and, also, to ensure the complete, accurate evacuation of the pump chamber.

### FIELD OF THE INVENTION

The invention relates generally to micropumps for drug infusion and more specifically to an engine design for a micropump with improved safety, reliability, and accuracy by employing a chamber design that includes an arrangement of the diaphragm and check valves that avoids the unintentional or undesirable release of fluid, which will usually be a medication for a patient, from a reservoir holding the fluid.

### BACKGROUND OF THE INVENTION

Diabetes mellitus is a chronic metabolic disorder caused by an inability of the pancreas to produce sufficient amounts of the hormone insulin so that the metabolism is unable to provide for the proper absorption of sugar and starch. This failure leads to hyperglycemia, i.e. the presence of an excessive amount of glucose within the blood plasma. Persistent hyperglycemia causes a variety of serious symptoms and life threatening long term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to always maintain the level of blood glucose within normal limits. Such glycemic control is achieved by regularly supplying external insulin to the body of the patient to thereby reduce the elevated levels of blood glucose.

External insulin was commonly administered by means of typically one or two injections of a mixture of rapid and intermediate acting insulin per day via a hypodermic syringe. While this treatment does not require the frequent estimation of blood glucose, it has been found that the degree of glycemic control achievable in this way is suboptimal because the delivery is unlike physiological insulin production, according to which insulin enters the bloodstream at a lower rate and over a more extended period of time. Improved glycemic control may be achieved by the so-called intensive insulin therapy which is based on multiple daily injections, including one or two injections per day of long acting insulin for providing basal insulin and additional injections of rapidly acting insulin before each meal in an amount proportional to the size of the meal. Although traditional syringes have at least partly been replaced by insulin pens, the frequent injections are nevertheless very inconvenient for the patient

Substantial improvements in diabetes therapy have been achieved by the development of the insulin infusion pump relieving the patient of the daily use of syringes or insulin pens. The insulin pump allows for the delivery of insulin in a more physiological manner and can be controlled to follow standard or individually modified protocols to give the patient a better glycemic control over the course of a day.

Infusion pumps can be constructed as an implantable device for subcutaneous arrangement or can be constructed as an external device with an infusion set for subcutaneous infusion to the patient. External infusion pumps are mounted on clothing, hidden beneath or inside clothing, or mounted on the body. Implanted pumps are controlled by a remote device. Most external infusion pumps are controlled through a built-in user interface, but control via a remote controller is available for some pump systems. Some pump systems use both a built-in pump user interface and a remote controller.

Regardless of the type of infusion pump, blood glucose monitoring is still required for glycemic control. For example, delivery of suitable amounts of insulin by the insulin pump requires that the patient frequently determines his or her blood glucose level and manually input this value into the remote device or into the built in user interface for some external pumps, which then calculates a suitable modification to the default or currently in use insulin delivery protocol, i.e. dosage and timing, and subsequently communicates with the insulin pump to adjust its operation accordingly. The determination of blood glucose concentration is performed by means of a suitable battery-operated measuring device such as a hand-held electronic meter which receives blood samples via enzyme-based test strips and calculates the blood glucose value based on the enzymatic reaction.

The meter device is an integral part of the blood glucose system and integrating the measuring aspects of the meter into an external pump or the remote of a pump is desirable.

Integration eliminates the need for the patient to carry a separate meter device, and it offers added convenience and safety advantages by eliminating the manual input of the glucose readings.

Current devices fail to meet all of the needs of diabetics, however, since many devices are inconveniently large and may not be easily or comfortably worn on the body. Devices that affix to the skin, or patch pumps, may be unreliable, as well, due to the difficulties of manufacturing micro-pumps capable of delivering precise quantities of insulin from a small, flexible reservoir that is desirable to use in devices that are designed to wear under clothing or by active, athletic persons.

### BRIEF DESCRIPTION OF THE FIGURES

FIG. 1A illustrates a syringe pump. FIG. 1B illustrates a micro diaphragm pump, according to an embodiment of the present invention.

FIGS. 2A through 2D illustrate a micro diaphragm pump, and its sequence of use, according to an embodiment of the present invention.

FIGS. 3A through 3G illustrate a micro diaphragm pump, and its sequence of use, according to an embodiment of the present invention.

FIG. 4 is an exploded view of a micro diaphragm pump, according to an embodiment of the present invention.

FIG. 5 is an exploded, partial assembly view of a micro diaphragm pump, according to an embodiment of the present invention.

FIG. 6A is an assembly view of a micro diaphragm pump, according to an embodiment of the present invention. FIG. 6B is a cross sectional view of the micro diaphragm pump illustrated in FIG. 6A.

FIGS. 7A through 7C illustrate a micro diaphragm pump, and its sequence of use, according to an embodiment of the present invention.

FIGS. 8A and 8B illustrate a spring and an assembly of springs, according to an embodiment of the present invention.

FIGS. 9 through 30 are graphs that illustrate the performance of micro diaphragm pumps, according to embodiments of the present invention.

FIGS. 31 and 32 illustrate sensor measurements taken during operation of micro diaphragm pumps, according to embodiments of the present invention.

FIGS. 33 and 34 illustrate micro pump status as a function of inlet valve sensor measurements, outlet valve sensor measurements, and actuator sensor measurements, according to embodiments of the present invention.

### DETAILED DESCRIPTION OF THE FIGURES

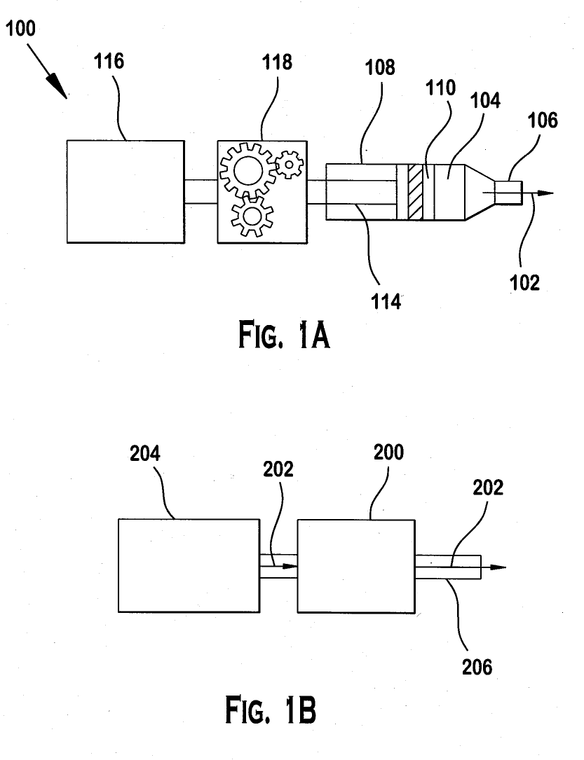

As illustrated in FIG. 1A, a syringe pump 100 typically includes a motor 116, a lead screw 118, a plunger 114, a syringe barrel 108, and a piston 110. In use, motor 116 turns lead screw 118, which is connected to plunger 114. As plunger 114 pushes against piston 110, infusion liquid 102 is forced from reservoir 104 through outlet 106. While syringe pumps 100 are safe and accurate, they are relatively large and expensive. In the present invention, illustrated in FIG. 1B, micro diaphragm pump 200 can be used to pump infusion liquid 202 directly from reservoir 204 to outlet 206, eliminating the need for a bulky lead screw, plunger, syringe barrel, and piston. Micro diaphragm pump 200 is often referred to as a direct pump because its mechanism makes direct contact with infusion liquid 202. Micro diaphragm pumps 200 are smaller and less expensive than syringe pumps 100, and are therefore less conspicuous and costly to the user.

Micro diaphragm pumps 200 are designed to meet numerous requirements. In terms of accuracy and delivery volume, micro diaphragm pumps 200 are typically designed to deliver at least ±5% accuracy at both very low flow rates (such as 0.5 microliters/hr) and very high flow rates (such as 100 microliters/min). In embodiments of the present invention, sensors are often used to control and verify delivery volume from micro diaphragm pumps 200. In terms of safety, embodiments of the present invention are designed in such a way as to minimize errors in volumetric delivery of infusion liquid 202. Micro diaphragm pumps 200 are designed in to minimize over-delivery and under-delivery of infusion liquid 202. In some embodiments of the present invention, micro diaphragm pumps 200 include sensors that rapidly detect occlusions in outlet 206, or in infusion lines or cannulas that may be connected to outlet 206. In addition, micro diaphragm pumps 200 are often protected from external interferences, such as electromagnetic, electrostatic, temperature variations, and physical impact. Micro diaphragm pumps 200 are designed to be reliable, since they are typically used 24 hours a day. Micro diaphragm pumps 200 are designed to withstand daily wear and tear, physical abuse, and even submersion in water, while still performing to specification. Micro diaphragm pumps 200, as embodied by the present invention, are considerably smaller than syringe pumps 100. In many embodiments, micro diaphragm pumps 200 are at least 50-70% smaller in size compared to syringe pumps 100. Because micro diaphragm pumps 200 are so small, it is possible to pump infusion liquid from multiple reservoirs, while maintaining smaller size than syringe pumps. In addition, when initially filling micro diaphragm pumps 200, it is possible to prime the pump, infusion lines, and connecting channels, removing bubbles that can adversely affect the accuracy of infusion. Micro diaphragm pumps 200 are easy to use, including the steps of filling, priming, connecting infusion sets, connecting cannulas and reservoirs, and attaching micro diaphragm pumps 200 to the user's body.

In the present invention, micro diaphragm pumps are described that meets these requirements. Micro diaphragm pumps of this invention can be used to infuse a variety of compounds, including cellular suspensions, solutions containing DNA, and pharmaceutical formulations. Compounds infused by micro diaphragm pumps of the present invention can be used in the treatment of conditions such as Parkinson's disease, epilepsy, chronic pain, immune system disorders, inflammatory diseases, obesity, and diabetes. Infused compounds include pharmaceutical formulations such as insulin, and GLP-1 drugs (such as Symlin, Byetta, etc). In the present invention, micro diaphragm pumps can be made using low cost, high volume manufacturing methods, including lamination, hot embossing, injection molding, and ultrasonic welding. Many different plastics can be used to achieve desired chemical and mechanical properties. Other materials, such as metal, can be used as well. In some embodiments of the present invention, metal is integrated with plastic components to produce features such as springs and electrical contacts. Thin polymer or metal layers can be laminated with thicker layers to produce moveable diaphragms and valves. In other embodiments of the present invention, components such as check valves, fluid flow channels, and diaphragms combine to form a single structure, allowing for simple manufacturing, reduced dead volume, and improved resolution and accuracy.

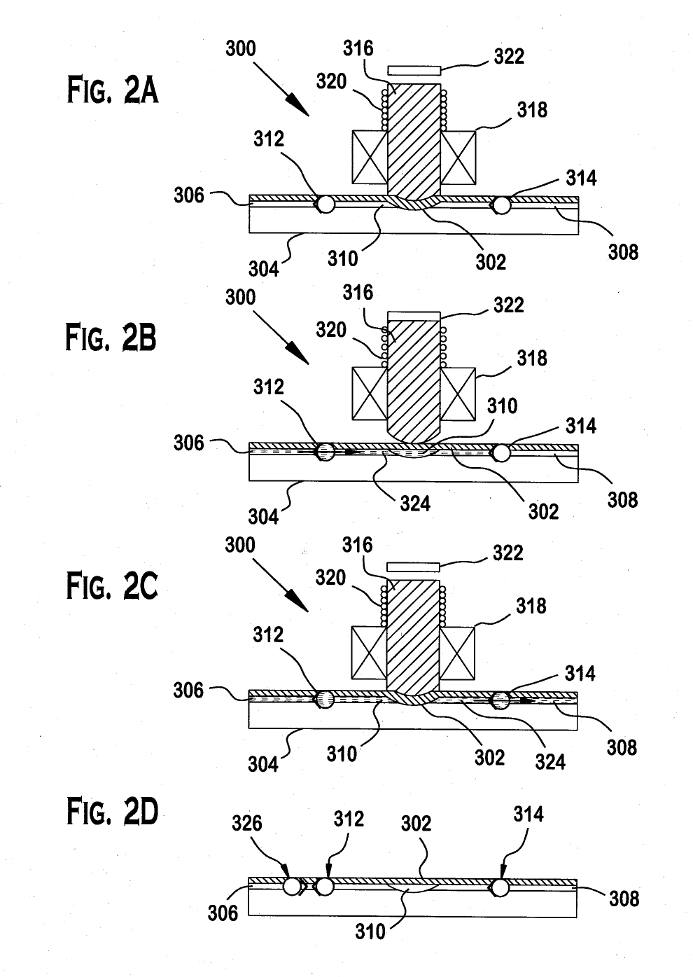

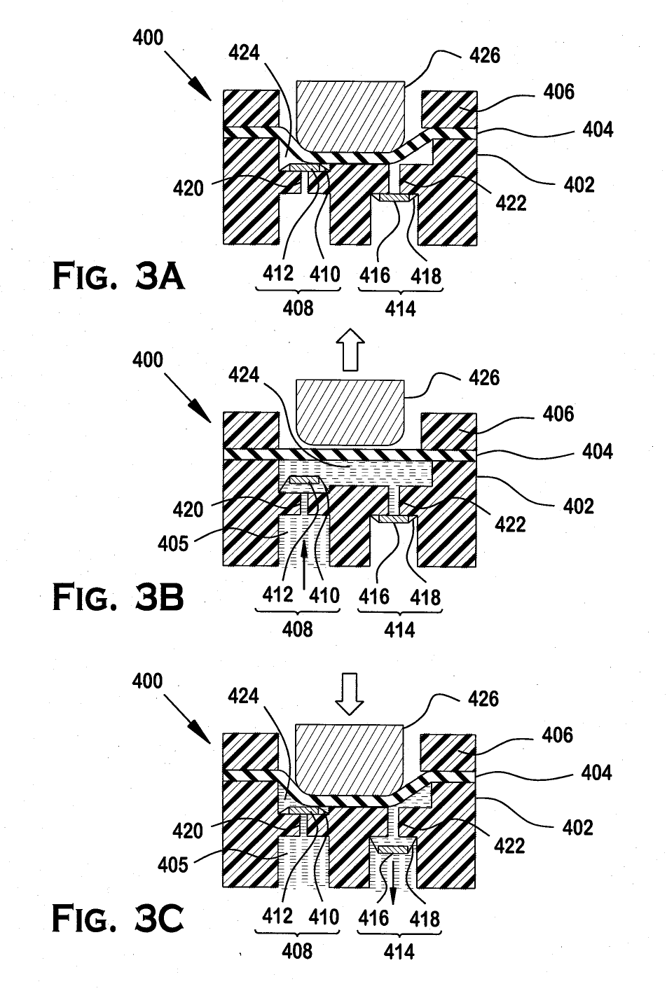

FIGS. 2A-2D illustrate embodiments of the present invention. Micro diaphragm pump 300 includes diaphragm 302, substrate 304, inlet channel 306, outlet channel 308, pump chamber 310, inlet check valve 312, outlet check valve 314, actuator 316, electromagnetic coil 318, actuator spring 320, and sensor 322. Inlet channel 306 can be connected to a reservoir, which is not shown, while outlet channel 308 can be connected to infusion lines and a cannula, which are not shown. The reservoir can be flexible or collapsible, as in the case of a plastic bag or pouch, or can be rigid, as in the case of a syringe or tube. Actuator 316 moves up and down, making contact with diaphragm 302, and forcing most of the infusion liquid from pump chamber 310. As illustrated in FIGS. 2A-2C, actuator 316 is enclosed by actuator spring 320 and electromagnetic coil 318, which impart up and down motion to actuator 316. Actuator 316 can be used with or replaced by other elements, such as a DC motor, a piezoelectric actuator, a thermopneumatic actuator, a shape memory alloy actuator, a bimetallic strip, an ion conductive polymer film, or other components that impart up and down motion to diaphragm 302. In some embodiments, diaphragm 302 extends beyond pump chamber 310 and forms the top layer of micro diaphragm pump 300. Diaphragm 302 can include an electrically conductive coating that forms electrical contact or capacitive coupling between diaphragm 302, substrate 304, actuator 316, and/or infusion liquid 324. In FIG. 2A, micro diaphragm pump 300 has yet to be used, actuator 316 is in its normally down position, and there is no infusion liquid in inlet channel 306, pump chamber 310, or outlet channel 308. Inlet channel 306, pump chamber 310, and outlet channel 308 are initially filled with air. In FIG. 2B, actuator 316 is in an upward position, infusion liquid has been drawn through inlet channel 306 into pump chamber 310, and outlet check valve 314 is closed. Infusion liquid flows through inlet check valve 312 because a drop in pressure is created in pump chamber 310 as actuator 316 moves up. As a drop in pressure is created in pump chamber 310, a pressure differential is created across inlet check valve 312, forcing it to open. In FIG. 2C, actuator 316 presses down on diaphragm 302, increasing the pressure in pump chamber 310. As pressure increases in pump chamber 310, inlet check valve 312 closes, and outlet check valve 314 opens, allowing flow of infusion liquid 324 from pump chamber 310 through outlet check valve 314 and outlet channel 308. A micro bolus of infusion liquid, equivalent to the volume displaced from pump chamber 310, is delivered through infusion lines connected to outlet channel 308. Although most of infusion liquid 324 is displaced from pump chamber 310, a small amount of infusion liquid 324 is typically left behind. The sequence in FIGS. 2B and 2C is repeated, until the desired volume of infusion liquid 324 is delivered. The shot size, or minimum deliverable volume, is approximately equal to the volume of infusion liquid 324 that is displaced from pump chamber 310 during the down stroke of actuator 316. Larger volumes are delivered by cycling micro diaphragm pump 300 multiple times. Various basal rates can be achieved by changing the up and down frequency of actuator 316.

Actuator spring 320 biases actuator 316 to the down position, while activating electromagnetic coil 318 lifts actuator 316 to the up position, elongating actuator spring 320. This “normally closed” configuration prevents infusion liquid 324 from inadvertently migrating from a reservoir through inlet channel 306 and outlet channel 308, as can happen in the event of sudden pressure rise in the reservoir or sudden pressure drop at outlet channel 308. Another safety feature associated with this configuration is the fact that electromagnetic coil 318 must be pulsed on and off for micro diaphragm pump 300 to operate. If power is accidentally applied to electromagnetic coil 318 in a continuous (rather than pulsed) manner, actuator 316 will remain in an up position, and infusion liquid 324 will not be forced from pump chamber 310. In embodiments of the present invention, solenoids and DC motors can be used as actuators, and are appealing because they produce large forces, resulting in consistent delivery even under conditions of variable backpressure, which can occur when encountering occlusion or scar tissue at the infusion site. The size of pump chamber 310 inherently limits the amount of infusion liquid that is delivered in a single cycle, relaxing engineering constraints on the travel distance and force produced by the actuator 316. In some embodiments of the present invention, sensors 322 are used to indirectly detect occlusions and siphoning errors, while in other embodiments encoders are used to determine the position of the actuator 316.

Actuator 316 can be part of a durable, reusable system, or can be part of a disposable system. A solenoid, DC motor, or piezoelectric based actuator 316 can be included in a durable system, along with electronics and a flexible membrane that protects durable components from ingress of water and debris, while allowing actuator 316 to interact with diaphragm 302. In embodiments of the present invention where a protective membrane is used, electrical contact between the durable and disposable components is optional. In embodiments of the present invention where actuator 316 is housed with the disposable components, other actuators can be used, such as those based on thermopneumatic, shape memory, and piezoelectric components.

In some embodiments of the present invention, sensor 322 can include a force sensor, contact sensor, or position sensor that works in conjunction with actuator 316. Sensor 322 can detect motion of actuator 316, and confirms that micro diaphragm pump 300 is operating as expected. If actuator 316 is not moving when it should, sensor 322 will detect the problem and an alarm will be activated, alerting the user to the error condition. Encoders and force sensors can be used in conjunction with actuator 316 to verify motion, to detect bubbles in pump chamber 310, and to detect occlusions in outlet channel 308 (or in infusion lines and cannulas). Bubbles in pump chamber 310 can reduce force at sensor 322, while occlusions can increase force at sensor 322. In other embodiments of the present invention, an electrical contact can be included on the surface of diaphragm 302, and can create an electrical switch when contact is made between actuator 316 and diaphragm 302. The electrical switch can be used to verify motion of actuator 316.

As mentioned previously, a reservoir is typically connected to inlet channel 306. An error mode can occur if the pressure in the reservoir is suddenly increased to unusually high pressures while actuator 316 is in the up position. If the pressure in the reservoir is high enough, infusion liquid 324 will overcome the backpressure of inlet check valve 312 and outlet check valve 314, causing flow through the pump, even when it is off. To overcome this error, some embodiments of the present invention include an over-pressure check valve 326, as illustrated in FIG. 2D. Over-pressure check valve 326 is oriented in an opposite direction to inlet check valve 312. Over-pressure check valve 326 allows infusion liquid 324 to pass when the reservoir is at normal pressure, but closes when the reservoir is at unusually high pressure. The pressure required to close over-pressure check valve 326 is greater than the pressure encountered during normal operation, when actuator 316 is in the up position and a slight drop in pressure has been created in pump chamber 310. If the pressure in the reservoir becomes unusually high (from an impact or from a change in airplane cabin pressure, for example), over-pressure check valve 326 will seal, preventing inadvertent flow of infusion liquid 324. Over-pressure check valve 326 can ensure that there is no delivery of infusion liquid 324 at abnormal reservoir pressures. If over-pressure check valve 326 seals, a drop in pressure may form in pump chamber 310 when actuator 316 moves up, and diaphragm 302 will typically stay in the down position, as illustrated in FIG. 2A. In embodiments where an electrical contact has been included in diaphragm 302, the electrical switch between actuator 316 and diaphragm 302 will stay open when diaphragm 302 stays in the down position and actuator 316 is up, and an alarm can be raised to alert the user. In other embodiments of the present invention, active valves, rather than check valves, are used to prevent flow from an over pressurized reservoir. Active valves rely on direct physical contact with an actuator to close, while check valves rely upon pressure differential across the valve to close. Active valves are typically more complicated than check valves, however, and in some cases require more sophisticated actuation.

Micro diaphragm pumps, according to the present invention, are a type of positive displacement pump. In positive displacement pumps, a pump chamber is filled then emptied by action of the pump. A distinct advantage of micro diaphragm pumps (and positive displacement pumps, in general) is that they can pump gas as well as liquid, if the compression ratio is high enough. The compression ratio is the volume displaced during the actuator down stroke divided by the volume of the pump chamber. Using a micro diaphragm pump is particularly advantageous when priming the pump, since air is expelled from the pump (and its inlet and outline lines) during priming. Micro diaphragm pumps are easy for a user to set up because they can pump air and infusion liquid. Centrifugal pumps, on the other hand, rely upon shear between an impeller and the liquid being pumped. Centrifugal pumps work better with liquid than with air, and are more difficult to set up.

As mentioned previously, a variety of methods can be used to fabricate micro diaphragm pumps, according to the present invention. Thin polymer and metal films can be laminated together to form a micro diaphragm pump. Layers of thermally activated adhesives can be used to laminate the films together. Check valves can include springs made from metal or plastic sheets. Check valve springs can be biased to create particular cracking and sealing pressure. Bias can be varied by controlling the relative position of the check valve and the surface against which it seats. Check valve springs can be made by chemically etching metal sheet or foil, or by cutting or injection molding plastics. Pump chamber volume can be established by the thickness of the metal and/or polymer and adhesive films. If necessary, the wetted surfaces of the pump can be coated with a polymer (such as parylene), to improve compatibility with infusion liquids. Ultrasonic welding, or other bonding methods, can be used instead of, or in addition to, thermally activated adhesives.

Compatibility with the infusion liquid is a particularly important requirement of micro diaphragm pumps of the present invention. In many embodiments, the infusion liquid is in direct contact with many parts of the pump. Infusion liquid can stick to wetted pump surfaces, and can be modified by chemical and/or physical interaction. In some embodiments of the present invention, wetted pump components are made out of biocompatible materials, such as polypropylene. In other embodiments, wetted pump components are coated with biocompatible materials such as paralyne, PEG, PAA, PVP, and/or polyelectrolyte. Biocompatible materials minimize adsorption of infusion liquid, and its degradation. Alternatively, pump components can be machined or injection molded using biocompatible polymers, such as PMMA, polycarbonate, polycyclic olefin, polystyrene, polyethylene, or polypropylene.

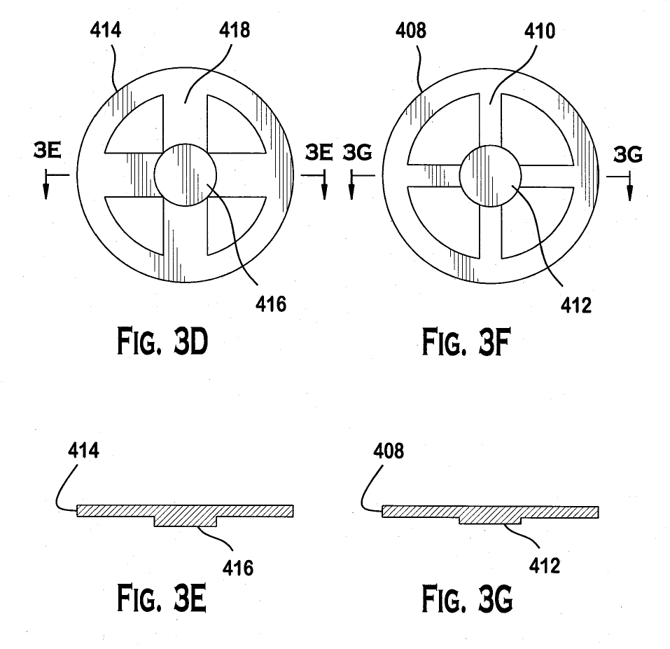

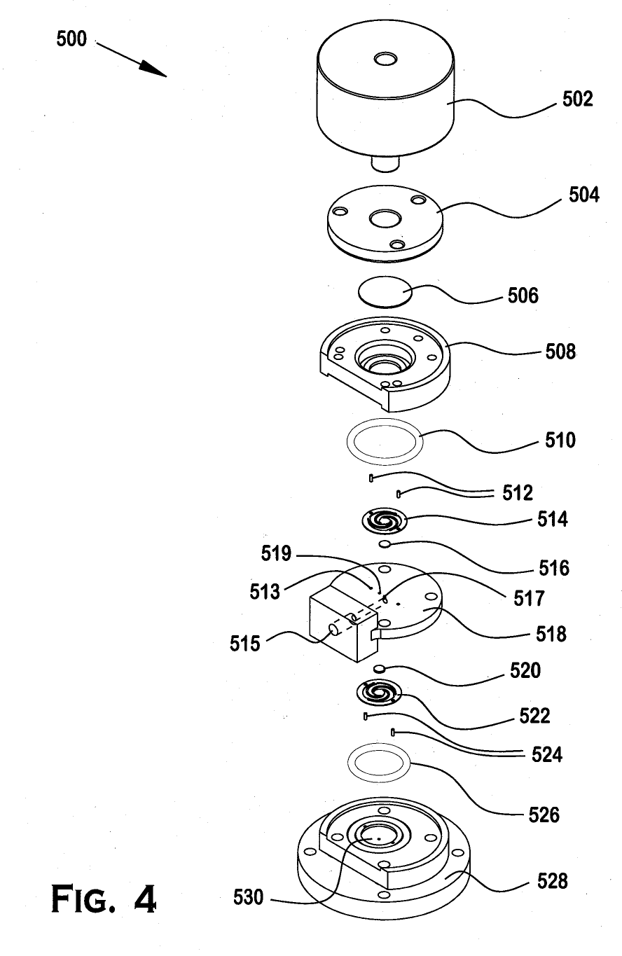

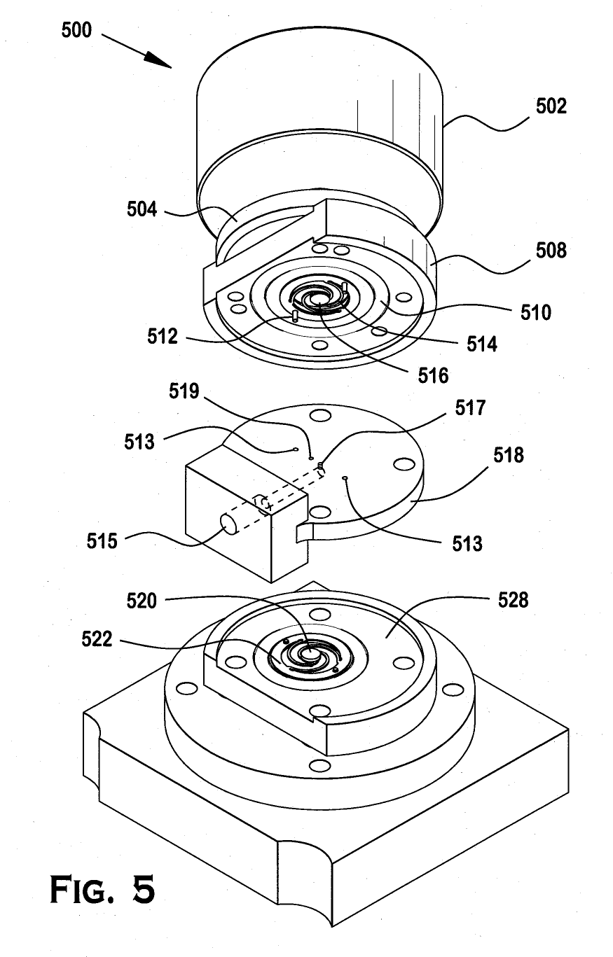

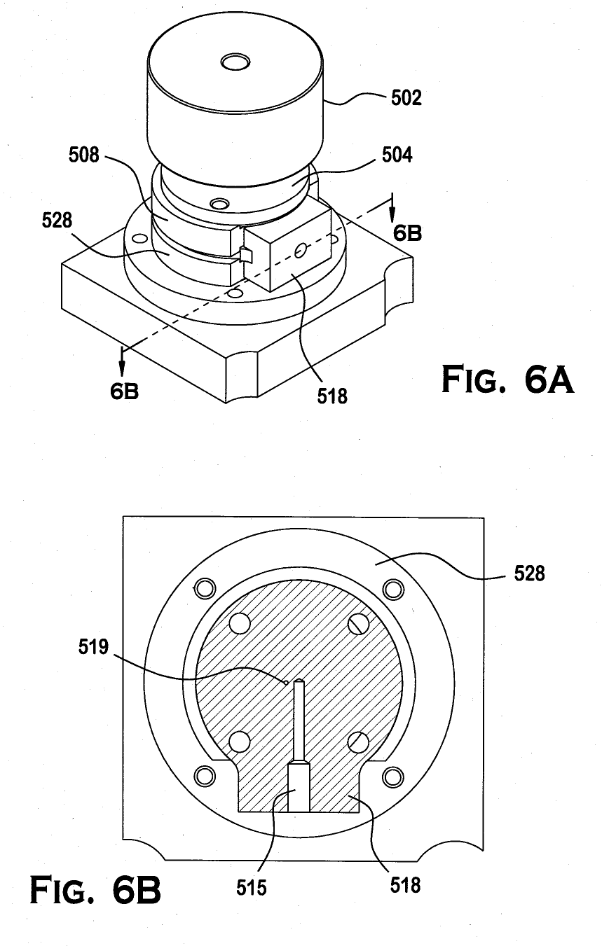

FIGS. 3A-3G illustrate an alternative embodiment of the present invention. Micro diaphragm pump 400 includes valve seat plate 402, diaphragm 404, diaphragm clamp 406, inlet check valve 408, outlet check valve 414, inlet channel 420, outlet channel 422, and actuator 426. Diaphragm clamp 406 fastens diaphragm 404 to valve seat plate 402, forming pump chamber 424. Inlet check valve 408 includes inlet spring 410 and inlet disk 412, while outlet check valve 414 includes outlet disk 416 and outlet spring 418. In FIG. 3A, micro diaphragm pump 400 is empty, and actuator 426 is in its down position. In the down position, actuator 426 pushes against diaphragm 404, making direct contact with inlet check valve 408. While actuator 426 and diaphragm 404 are in direct contact with inlet check valve 408, they provide additional sealing force between inlet check valve 408 and inlet channel 420, actively closing check valve 408. This is useful in preventing inadvertent flow through micro diaphragm pump 400 when the pump is off. Returning to FIG. 3A, before it has been used, micro diaphragm pump 400 contains no infusion liquid 405, and inlet check valve 408 and outlet check valve 414 are closed. In FIG. 3B, a pump cycle has begun. Actuator 426 is in the up position, and diaphragm 404 has moved upward, creating a drop in pressure in pump chamber 424. The drop in pressure in pump chamber 424 creates a pressure differential across inlet channel 420, stretching inlet spring 410 and moving inlet disk 412 upward. This allows infusion liquid 405 to flow through inlet channel 420, around inlet disk 412, and into pump chamber 424. Meanwhile, the drop in pressure in pump chamber 424 causes additional sealing force across outlet channel 422, pushing outlet disk 416 against outlet channel 422, and preventing flow of infusion liquid 405 from pump chamber 424 through outlet channel 422. In FIG. 3C, actuator 426 returns to a down position, pushing infusion liquid 405 out of pump chamber 424. As actuator 426 moves downward, pressure in pump chamber 424 increases, causing inlet disk 412 to seal against inlet channel 420, and pushing outlet disk 416 away from outlet channel 422. As outlet disk 416 moves away from outlet channel 422, infusion liquid 405 moves from pump chamber 424, around outlet spring 418 and outlet disk 415, and through outlet channel 422, completing a pump cycle. Actuator 426 and diaphragm 404 displace most of infusion liquid 405 from pump chamber 424. If desired, the steps illustrated in FIGS. 3B and 3C can be repeated to deliver additional infusion liquid 405. FIGS. 3D-3G are plan and cross sectional views of outlet check valve 414 and inlet check valve 408. In FIGS. 3D and 3E, outlet check valve 414 includes outlet spring 418 and outlet disk 416. Outlet spring 418 determines the spring or force constant of outlet check valve 414. If outlet spring 418 is wide, short in length, and/or thick, the spring or force constant of outlet check valve 414 increases. If outlet spring 418 is narrow, long in length, and/or thin, the spring or force constant of outlet check valve 414 decreases. Higher spring or force constant leads to higher opening (or cracking) pressure, while lower spring or force constant leads to lower cracking pressures. In FIGS. 3F and 3G, inlet check valve 408 includes inlet spring 410 and inlet disk 412. In some embodiments of the present invention, outlet spring 418 and inlet spring 410 are different in shape. This leads to different cracking pressures between outlet check valve 414 and inlet check valve 408. This can improve the performance of micro diaphragm infusion pump 400, by maximizing the sealing force across outlet check valve 414 and inlet check valve 408, while still allowing flow of infusion liquid 405 at appropriate times in the pump cycle. Another way to create a difference in cracking pressure between outlet check valve 414 and inlet check valve 408 is to vary their bias force in the closed position. This can be achieved by varying the thickness of outlet disk 416 and inlet disk 412. The thickness of outlet disk 416 and inlet disk 412 establish the extent to which outlet spring 418 and inlet spring 410 are stretched when closed. In embodiments of the present invention, outlet spring 418 and inlet spring 410 are made out of metal or plastic, and typically follow Hooke's law. Hooke's law states that the force with which a spring closes is linearly proportional to the distance from its relaxed position. By changing the thickness of outlet disk 416 and/or inlet disk 412, the distance from its relaxed position is changed, increasing or decreasing its closing force. FIG. 3E illustrates a thick outlet disk 416, while FIG. 3G illustrates a thin inlet disk 412. A thick outlet disk 416 leads to greater closing force, while a thin inlet disk 412 leads to less closing force.

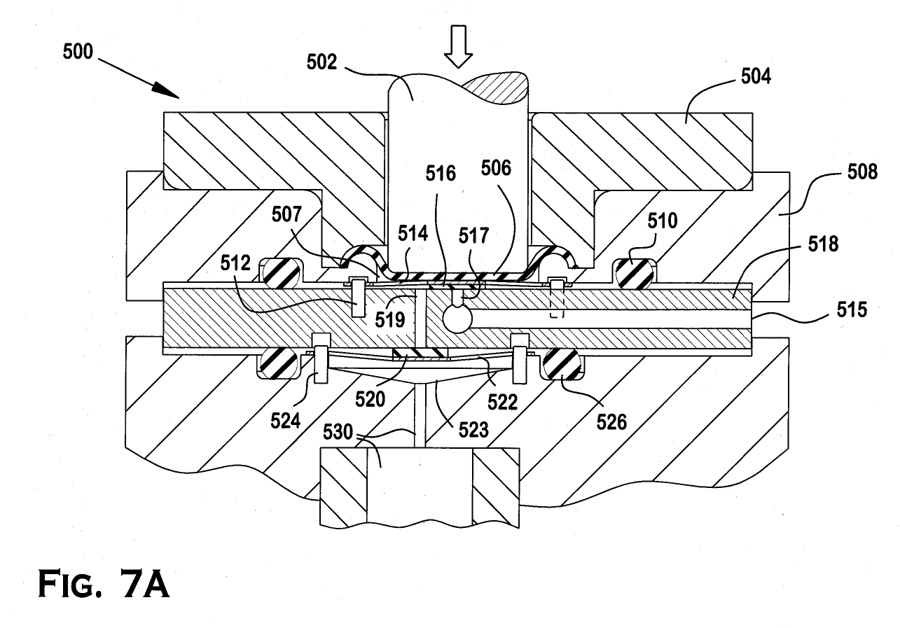

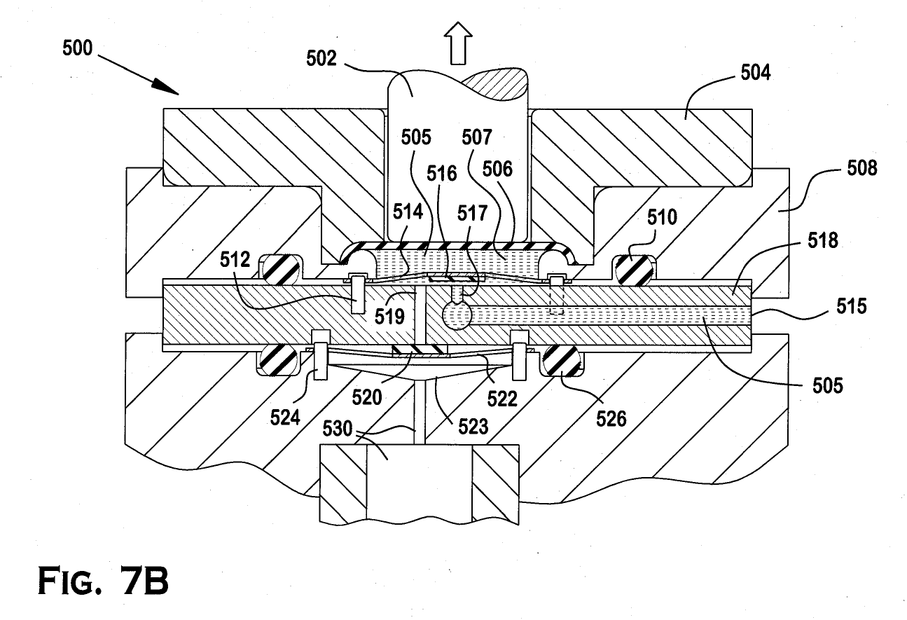

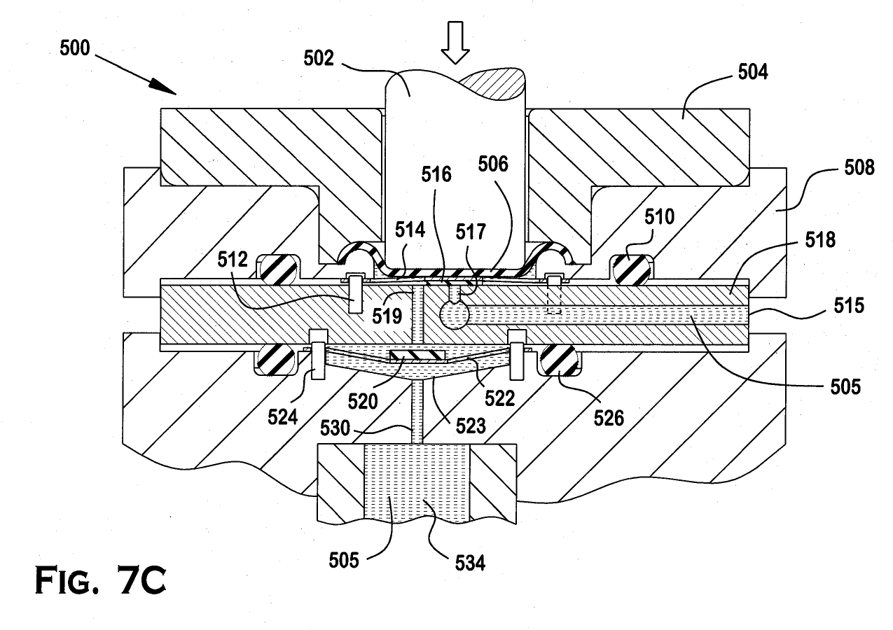

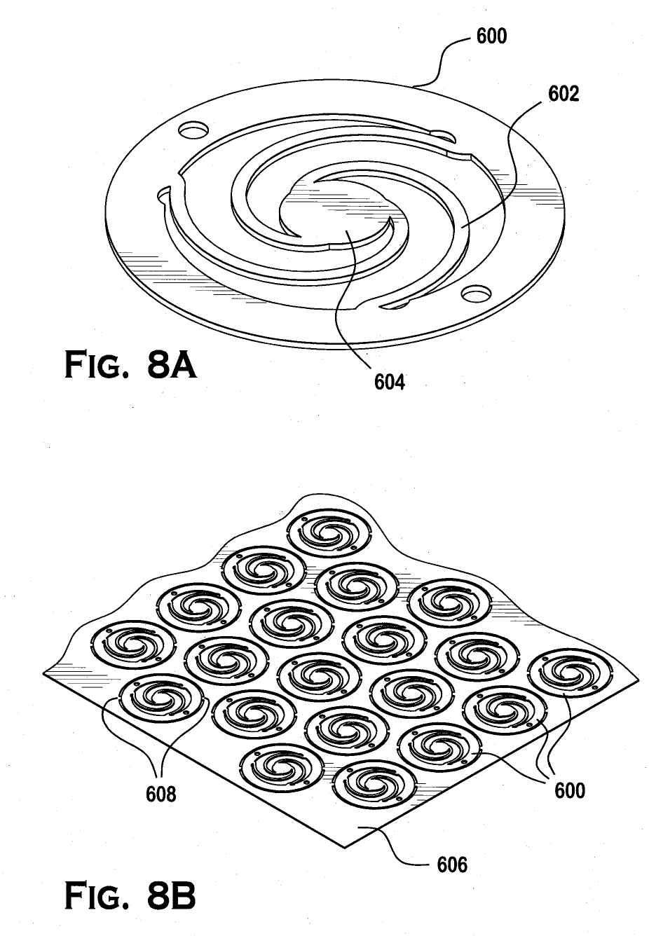

FIGS. 4-7 illustrate an alternative embodiment of the present invention. FIG. 4 illustrates an exploded view of a micro diaphragm pump. FIG. 5 illustrates an exploded, partially assembled view of the micro diaphragm pump that is illustrated in FIG. 4. FIG. 6A illustrates an assembled view, and FIG. 6B illustrates a cross sectional view of the micro diaphragm pump illustrated in FIGS. 4 and 5. FIGS. 7A through 7C are cross sectional views that illustrate flow of infusion liquid through the micro diaphragm pump illustrated in FIGS. 4-6.

In FIG. 4, micro diaphragm pump 500 includes actuator 502, diaphragm clamp 504, diaphragm 506, inlet housing 508, inlet seal 510, alignment pins 512, inlet spring 514, inlet disk 516, valve seat plate 518, outlet disk 520, outlet spring 522, alignment pins 524, outlet seal 526, and outlet housing 528. Valve seat plate 518 includes inlet port 515, inlet channel 517, and outlet channel 519. Valve seat plate 518 also includes alignment holes 513, which receive alignment pins 512. Outlet housing 528 includes outlet port 530. As a point of reference, inlet spring 514 and outlet spring 522 are about 6 mm in diameter, in some embodiments of the present invention. Actuator 502 can include any of the components previously mentioned in respect to other embodiments of the present invention. It can include springs or electromagnetic coils, as well as DC motors, cams, shape memory metals, or piezoelectric materials. Diaphragm clamp 504, seals diaphragm 506 against inlet housing 508, and partially defines the pump chamber (507 in FIG. 7B). Diaphragm 506 forms the upper layer of the pump chamber, and deflects when contacted by actuator 502, displacing most of the infusion liquid (505 in FIG. 7B) from pump chamber 507. Diaphragm 506 can be made of metal or plastic, as mentioned previously. When diaphragms 506 are made out of an elastic rubber, they conform particularly well, expelling nearly all of infusion liquid 505 from the pump chamber 507. This is particularly advantageous, and leads to greater compression ratios and better pump performance. When diaphragms 506 are made of metal, they spring back with great force when actuator 502 returns to an upward position. In some embodiments of the present invention, diaphragms 506 are made out of a metal spring covered with a thin sheet of elastic rubber, combining the spring back force of metal with the conformability of elastic rubber. Inlet housing 508 defines a portion of the pump chamber, and supports diaphragm 506. Diaphragm 506 is hermetically sealed between diaphragm clamp 504 and inlet housing 508. Inlet seal 510 is positioned between inlet housing 508 and valve seat plate 518, forming a hermetic seal between the pump chamber and the atmosphere. Inlet seal 510 can be in the shape of an o-ring, or in any other shape that provides a hermetic seal. In some embodiments of the present invention, inlet seal 510 and spring 514 can be combined into a single element. For example, a thermoplastic rubber can be insert molded around the edge of spring 514, decreasing the number of discrete components in micro diaphragm pump 500. Alignment pins 512 are inserted into alignment holes 513 and facilitate registration between various components of the diaphragm micro pump. Inlet spring 514 is sandwiched between inlet housing 508 and valve seat plate 518, and stretches up and down within the pump chamber. Inlet spring 514 may be fabricated using any of the methods described in respect to other embodiments of the present invention. In this embodiment of the present invention, inlet disk 516 is a separate component, but is physically attached to inlet spring 514. This allows inlet disk 516 to be made from different material than inlet spring 514. For example, inlet spring 514 could be made of stainless steel, while inlet disk 516 could be made of silicone rubber. Silicone rubber is much softer than stainless steel, and can form a more reliable seal with inlet channel 517. On the other hand, stainless steel has a greater spring or force constant, which leads to greater sealing force. By using separate components, the properties of inlet spring 514 and inlet disk 516 can be optimized. Inlet spring 514 and inlet disk 516 can be joined using a variety of methods, including adhesives, injection molding, and physical retaining features. Valve seat plate 518 is sandwiched between inlet housing 508 and outlet housing 528, forming hermetic seals via inlet seal 510 and outlet seal 526. Valve seat plate 518 includes inlet port 515 and inlet channel 517, through which infusion liquid flows from an external reservoir into the pump chamber. Inlet disk 516 seats against a smooth surface surrounding inlet channel 517, preventing flow through inlet channel 517 when appropriate. Valve seat plate 518 includes outlet channel 519, through which infusion liquid flows when pushed out of the pump chamber. Outlet disk 520 seats against a smooth surface surrounding outlet channel 519, preventing flow from the pump chamber when appropriate. As mentioned in respect to inlet spring 514 and inlet disk 516, outlet spring 522 and outlet disk 520 are separate components, allowing their physical properties to be optimized. They are attached using the methods mentioned previously. In some embodiments of the present invention, the smooth surface surrounding inlet channel 517 and outlet channel 519 is made out of a soft material, such as silicone rubber. This makes the smooth surface surrounding inlet channel 517 and outlet channel 519 conformable, and improves its ability to form a tight seal with inlet disk 514 and outlet disk 520. In designs where the smooth surface surrounding inlet channel 517 and outlet channel 519 is made out of a soft material, inlet disk 516 and outlet disk 520 are optional, since inlet spring 514 and outlet spring 522 can form a direct seal with the soft material. Outlet seal 526 is similar to inlet seal 510, and forms a hermetic seal between valve seat plate 518 and outlet housing 528. Alignment pins 524 allow registration of various micro diaphragm components. Outlet housing 528 includes outlet port 530, through which infusion liquid flows when pushed out of the pump chamber.

FIG. 5 illustrates an exploded, partially assembled view of the micro diaphragm pump that is illustrated in FIG. 4. Actuator 502 is fastened to diaphragm clamp 504 and inlet housing 508. Diaphragm 506 (not shown) is sandwiched between diaphragm clamp 504 and inlet housing 508, forming a hermetic seal around the perimeter of diaphragm 506. Using alignment pins 512, inlet spring 514 and inlet disk 516 have been attached to inlet housing 508. As mentioned previously, inlet disk 516 is permanently attached to inlet spring 514. Valve seat plate 518 is shown in perspective, and is ready to be attached to inlet housing 508 and outlet housing 528. Valve seat plate 518 includes inlet port 515, which can be sized to accept Luer fittings. Valve seat plate 518 also includes inlet channel 517 and outlet channel 519, which pass completely through valve seat plate 518. The area around inlet channel 517 and outlet channel 519 is smooth, allowing inlet disk 516 and outlet disk 520 to form airtight seals around inlet channel 517 and outlet channel 519. Near the bottom of FIG. 5, outlet housing 528 has been attached to outlet spring 522 and outlet disk 520. As mentioned previously, outlet spring 522 and outlet disk 520 are permanently attached to each other. Outlet disk 520 forms an airtight seal as it presses against the smooth surface surrounding outlet channel 519.

FIG. 6A illustrates an assembled view, and FIG. 6B illustrates a cross sectional view of the micro diaphragm pump illustrated in FIGS. 4 and 5. In FIG. 6A, the components illustrated in FIG. 4 have been completely assembled. Although it is not shown in the drawing, screws can be used to fasten the components together. Actuator 502, diaphragm clamp 504, inlet housing 508, valve seat plate 518, and outlet housing 528, can be seen in the view illustrated by FIG. 6A. Inlet port 515 can be seen on the side of valve seat plate 518. FIG. 6B is a sectional view of FIG. 6A taken along line 6B-6B′. In FIG. 6B, valve seat plate 518 sits on top of outlet housing 528. Inlet port 515 enters the side of valve seat plate 518, ending near the center of valve seat plate 518. Outlet channel 519 passes through valve seat plate 518, as it approaches outlet housing 528.

FIGS. 7A through 7C are cross sectional views that illustrate flow of infusion liquid through the micro diaphragm pump illustrated in FIGS. 4-6. In FIG. 7A, micro diaphragm pump 500 has yet to be used, and there is no infusion liquid in any of its channels or chambers. Actuator 502 is in the down position, pressing diaphragm 506 and inlet disk 516 against inlet channel 517. This actively closes the inlet valve and prevents anything from flowing through inlet channel 517, even if the infusion reservoir (connected to inlet port 515, and not shown) is pressurized, or if there is siphoning. Outlet disk 520 presses against outlet channel 519 due to bias in outlet spring 522. In FIG. 7B, actuator 502 is raised to an upward position, allowing diaphragm 506 to relax, creating a drop in pressure in pump chamber 507. As the pressure in pump chamber 507 decreases, pressure in the inlet channel pushes against inlet disk 516 forcing it and inlet spring 514 into an upward position. As inlet disk 516 moves upward, infusion liquid 505 enters pump chamber 507. Meanwhile, lower pressure in pump chamber 507 increases the pressure difference across outlet disk 520, forcing outlet disk 520 against the smooth surface around outlet channel 519. This seals outlet channel 519, preventing infusion liquid 505 from leaving pump chamber 507. In FIG. 7C, actuator 502 has been moved to the downward position. As actuator 502 moves to the downward position, pressure in pump chamber 507 increases, and inlet disk 516 pushes against inlet channel 517, preventing flow through inlet channel 517. Initially, inlet disk 516 pushes against inlet channel 517 due to a pressure difference across inlet disk 516 and bias force caused by inlet spring 514. Eventually, diaphragm 506 makes direct contact with inlet disk 516, increasing the force with which inlet disk pushes against inlet channel 517. This provides a tight seal at inlet channel 517. Meanwhile, the increasing pressure in pump chamber 507 pushes against outlet disk 520 and outlet spring 522, forcing them away from outlet channel 519. As this happens, most of the infusion liquid 505 is forced from pump chamber 507 through outlet channel 519, and into outlet port 530. Each cycle of the pump (as illustrated in FIGS. 7B and 7C) dispenses a volume that is approximately equivalent to the volume of infusion liquid 505 displaced from pump chamber 507. If a volume greater than the volume of pump chamber 507 is desired, or if a continuous dispense rate is desired, the pump cycle is repeated. In the embodiment of the present invention illustrated in FIGS. 7A-7C, actuator 502 is in a downward position when micro diaphragm pump 500 is turned off. As mentioned previously, this provides additional force to seal inlet channel 517 with inlet disk 516. In other embodiments, actuator 502 is in an upward position when micro diaphragm pump 500 is turned off. In those embodiments, the bias of inlet spring 514 and outlet spring 522 provide force to seal inlet disk 516 against inlet channel 517, and to seal outlet disk 520 against outlet channel 519. Both embodiments of micro diaphragm pump 500 have been found to work well.

FIGS. 8A and 8B illustrate springs that can be used in embodiments of the present invention. The springs illustrated in FIGS. 8A and 8B can be used as either inlet springs or outlet springs, as described previously. In FIG. 8A, spring 600 includes elastic elements 602 and disk support 604. The shape and thickness of elastic elements 602 affect the force needed to stretch and relax spring 600. Disk support 604 can be attached to separate inlet or outlet disks, as described previously. This allows spring 600 and inlet or outlet disks to be made of different materials, and in different thicknesses, depending upon the application. Elastic elements 602 also allow disk support 604 to self align, when coupled with inlet or outlet disks. Self-alignment improves the seal between inlet and outlet disks and inlet and outlet channels. For instance, if the smooth surface around an inlet channel is not perfectly parallel with the sealing surface of an inlet disk, elastic elements in the inlet spring can twist, allowing the inlet disk to seat parallel to the smooth surface around the inlet channel. In addition, the diameter of the inlet or outlet disk can be much larger than the diameter of the inlet or outlet channel, allowing significant eccentricity while still forming a seal. FIG. 8B illustrates a sheet 606 of etched springs 600, as used in embodiments of the present invention. Springs 600 are chemically etched into 100 micron thick stainless steel, using a process that can run in either a batch or continuous fashion. Alternatively, springs 600 can be stamped in either batch or continuous mode. Springs 600 can remain attached to sheet 606 by tabs 608, lending themselves to automated inspection and assembly.

As mentioned previously, hard metals do not always form good seals when pressed against an inlet or outlet channel. In addition, in some embodiments of the present invention, inlet and outlet springs are flat, as illustrated in FIGS. 8A and 8B. For this reason, a soft inlet or outlet disk can be attached to disk support 604. Soft inlet or outlet disks conform to any surface irregularities and form good seals with inlet or outlet channels. In addition, inlet and outlet disks deflect elastic elements 602, causing bias and pre-tension, which also leads to better seals. Various methods can be used to attach inlet or outlet disks to disk supports 604, including adhesives, insert or over molding, and mechanical bonding using retaining features. In some embodiments, inlet or outlet disks are cut from silicone sheet, and glued to disk support 604 using silicone adhesives. In other embodiments, silicone rubber is dispensed as a droplet onto disk support 604, forming a solid inlet or outlet disk when cured. In further embodiments, thermoplastic or thermosetting rubber can be molded directly onto disk support 604 using insert molding techniques. Retaining features can be included in disk support 604, helping to keep cured silicone attached to disk support 604.

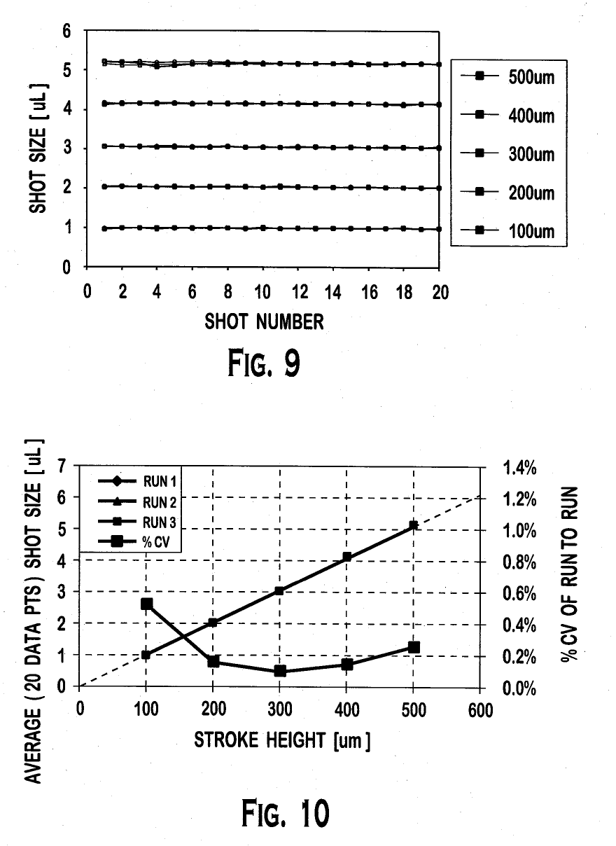

To determine the performance of micro diaphragm pumps of the present invention, a series of experiments were conducted. The results of the experiments are illustrated in FIGS. 9-30, and are described below. In many of the experiments, a motor moves the pump's actuator. This is referred to as automatic control. In other experiments, the actuator is moved by hand. This is referred to as manual control. In addition, some of the micro diaphragm pumps are configured in such a way that the actuators are in a down position when the pump is off. In a down position, the actuator pushes against the inlet spring and inlet disk, helping the disk to seal the inlet channel. This pump configuration is referred to as “active”. In other experiments, the micro diaphragm pumps are configured in such a way that the actuators are in an up position when the pump is off. In an up position, the actuator does not directly contact the inlet spring and inlet disk. Spring bias and the pressure differential across the inlet disk force the inlet disk against the inlet channel. Since there is no direct contact between the actuator and the inlet spring or inlet disk, this pump configuration is referred to as “passive”. As illustrated in the following Figures, active and passive configurations deliver excellent performance, although, active configurations provide additional sealing force when the pump is off. In all of the experiments described below, the infusion liquid is water. The dispensed volume, or “shot size”, was determined by pumping water onto an electronic balance, then mathematically converting mass to volume. The distance traveled by the actuator is referred to as “stroke height”, while the amount of time between one stroke and the next is referred to as “cycle time”.

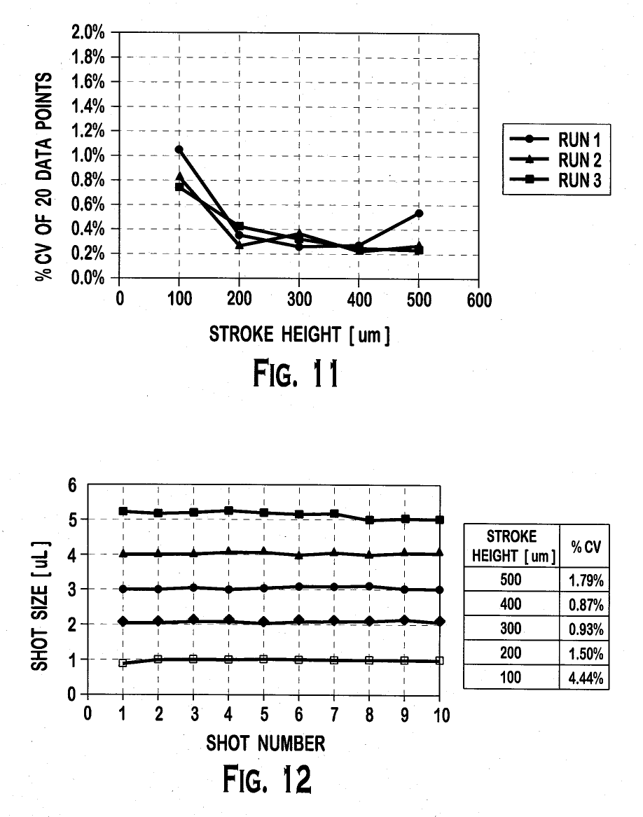

FIGS. 9 and 10 illustrate shot size as a function of stroke height for an automatically controlled, active, micro diaphragm pump. Stroke heights of 100, 200, 300, 400, and 500 microns result in shot sizes of approximately 1, 2, 3, 4, and 5 microliters, respectively. Twenty measurements were made at each stroke height, showing good reproducibility from shot to shot. In FIG. 10, shot size is plotted as a function of stroke height. FIG. 11 shows within pump shot-to-shot variability of less than 1%.

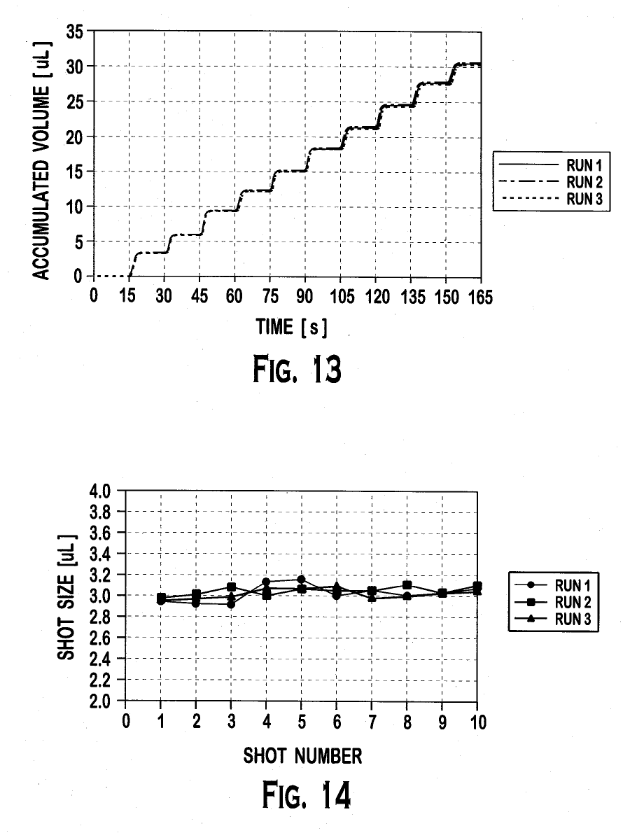

FIG. 12 illustrates shot size as a function of stroke height for a manually controlled, passive, micro diaphragm pump. Shot size variability is low, with coefficients of variation (% CV) of between 0.87 and 4.44%. FIG. 13 illustrates accumulated dispensed volume versus time, with three replicates. The replicates demonstrate good within pump reproducibility using manual control and a passive pump configuration. FIG. 14 illustrates individual shot sizes for the data illustrated in FIGS. 12 and 13. FIGS. 12-14 demonstrate that good precision and accuracy can be achieved with a manually controlled, passive, micro diaphragm pump.

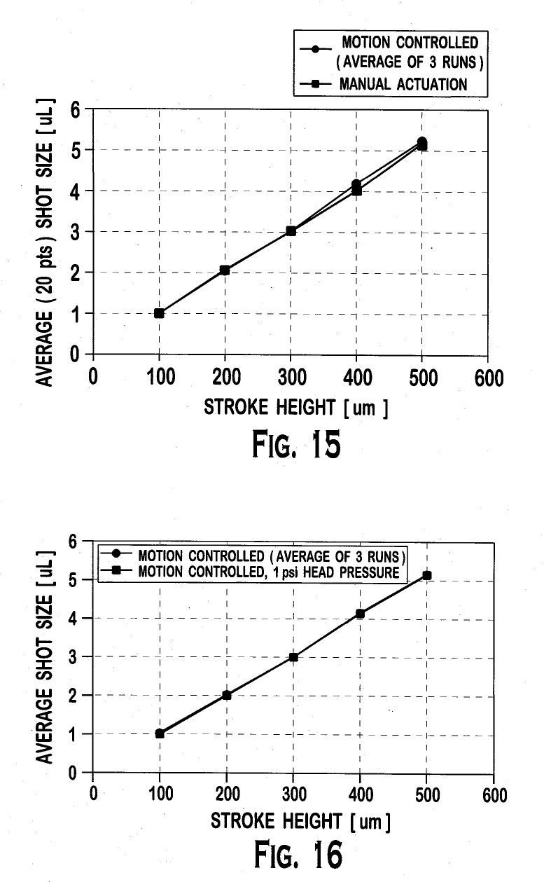

FIG. 15 illustrates average shot size as a function of stroke height for an automatically controlled, active, micro diaphragm pump, and for a manually controlled, passive, micro diaphragm pump. As can be seen in FIG. 15, shot size is consistent for both pumps. This result suggests that micro diaphragm pumps can be either automatically or manually controlled, and can be of an either active or passive configuration.

FIG. 16 illustrates the effect of backpressure on the performance of an automatically controlled, active, micro diaphragm pump. In this experiment, lowering the micro diaphragm pump below the level of the electronic balance created backpressure. As can be seen in FIG. 16, shot size as a function of stroke height was similar when pumping against 0 and 1 psi backpressures. This is an important result, in that a variety of backpressures can be encountered in everyday use.

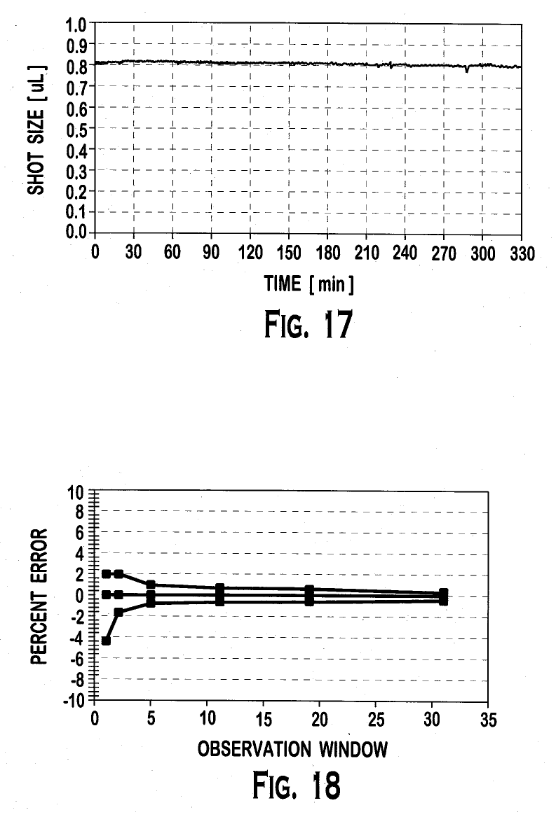

FIG. 17 illustrates shot size as a function of time, across many pump cycles. In this experiment, an automatically controlled, passive, micro diaphragm pump used a fixed stroke height of 300 microns. Cycle time was 1 minute, and the test lasted for 330 cycles.

FIG. 18 is a trumpet curve of the last 100 shots in FIG. 17. The target shot size was set to the average shot size, resulting in zero average error in the trumpet curve. The largest deviation from the average of any single shot is only 4% for 0.8 microliter shots. This demonstrates consistent shot size across many pump cycles.

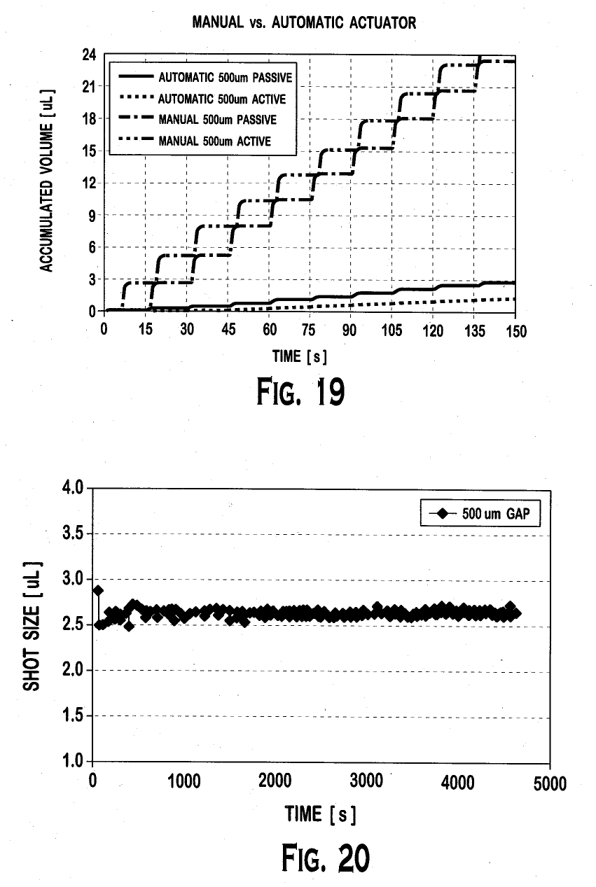

FIG. 19 illustrates accumulated volume as a function of time for the same micro diaphragm pump set up four different ways. First, the pump was automatically controlled with passive pump configuration. Next, the pump was automatically controlled with active pump configuration. Next, the pump was manually controlled with passive pump configuration. Finally, the pump was manually controlled with active pump configuration. In each case, the pump had a leaky inlet valve. Both manually controlled pumps performed well, despite the leaky inlet valve. Both automatically controlled pumps did not perform well. In this experiment, the actuator in manually controlled pumps moves much faster than the actuator in automatically controlled pumps. Because of this, pressure in the pump chamber increased very rapidly during the down stroke, helping to close the leaky inlet valve before infusion liquid could flow back through the inlet channel. This experiment demonstrates that stroke speed should be rapid, rather than slow.

In the following two experiments, a micro diaphragm pump is connected at its inlet to a pre-filled insulin cartridge. The pre-filled insulin cartridge was filled with water, rather than insulin. In this arrangement, the micro diaphragm pump draws water out of the pre-filled cartridge, creating a negative pressure that advances the syringe plunger, taking up the volume of water delivered by the pump. This type of cartridge is typically used in insulin pens and pumps that push on the syringe plunger to deliver insulin. Drawing fluid from the outlet of the syringe plunger is novel. For this approach to work, a micro diaphragm pump must generate a sufficient drop in pressure to advance the syringe plunger, overcoming static and dynamic friction.

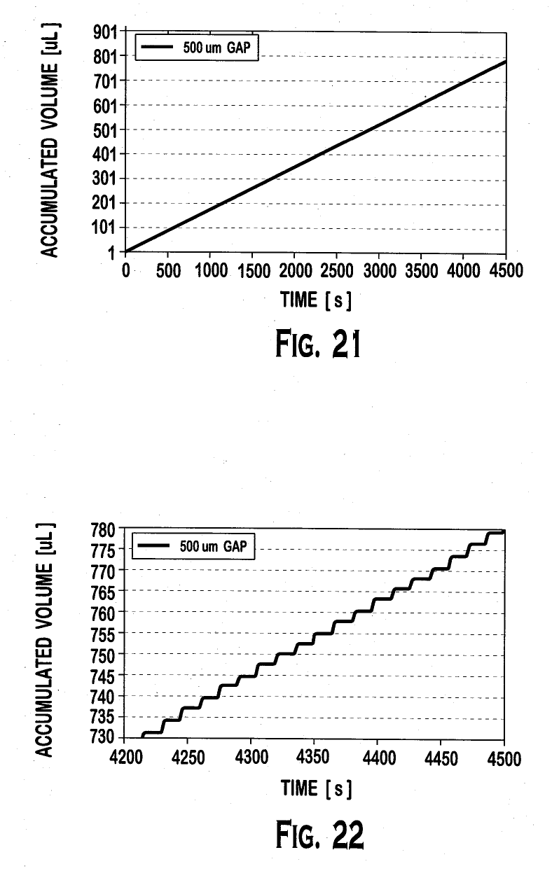

FIG. 20 illustrates shot size as a function of time for an automatically controlled, active, micro diaphragm pump connected at its inlet to a pre-filled insulin cartridge. A stroke height of 500 microns, and a cycle time of 15 seconds were used. The insulin cartridge was filled with water, rather than insulin. As shown in FIG. 20, average shot size was 2.6 microliters (equivalent in volume to 0.26 Units of U100 insulin), and the experiment lasted for 300 cycles. FIG. 21 illustrates accumulated volume as a function of time for the experiment illustrated in FIG. 20. As seen in FIG. 21 the micro diaphragm pump delivered linear performance throughout the experiment. FIG. 22 illustrates accumulated volume as a function of time during the last 300 seconds of the experiment. FIG. 22 suggests that the micro diaphragm pump delivers consistent shot size throughout the test. FIG. 23 is a trumpet curve for the last 100 data points of FIG. 20. The target shot size is set to the average shot size, resulting in zero average error. The maximum spread in shot size is ±2%, which is exceptionally low. This experiment demonstrates that micro diaphragm pumps of the present invention can accurately and precisely draw infusion liquid from the outlet of a pre-filled insulin cartridge, at large shot sizes.

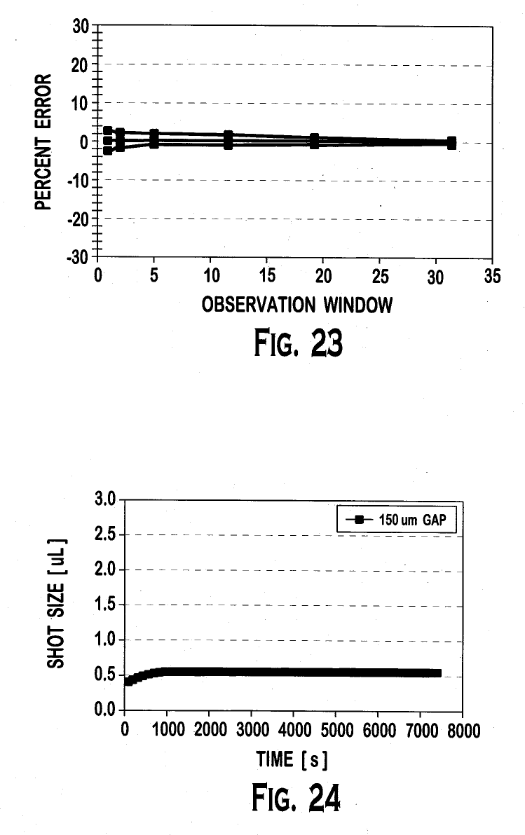

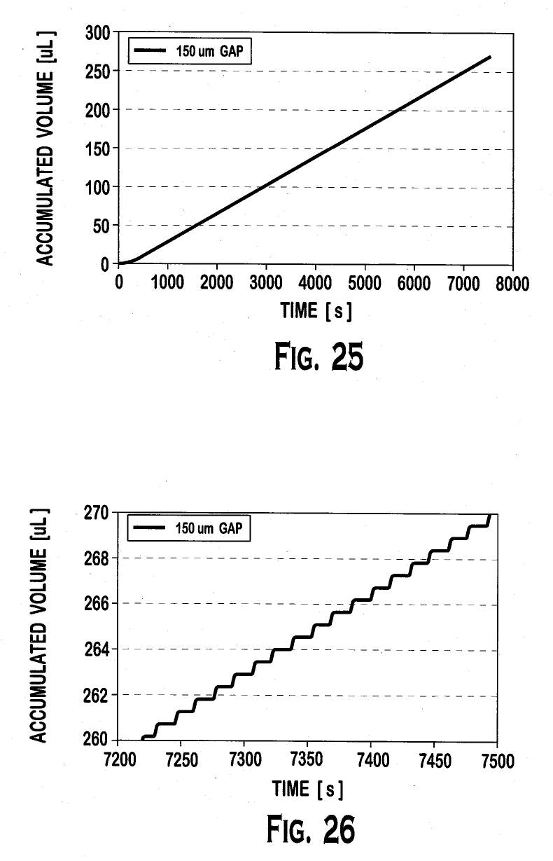

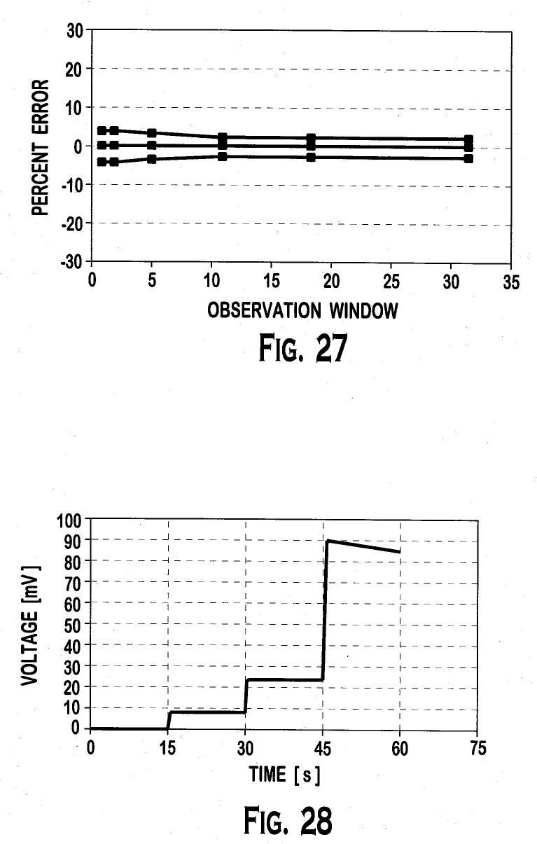

FIG. 24 illustrates shot size as a function of time for an automatically controlled, active, micro diaphragm pump connected at its inlet to a pre-filled insulin cartridge. A stroke height of 150 microns, and a cycle time of 15 seconds were used. The insulin cartridge was filled with water, rather than insulin. As shown in FIG. 24, average shot size was 0.5 microliters (equivalent in volume to 0.05 Units of U100 insulin), and the experiment lasted for 500 cycles. FIG. 25 illustrates accumulated volume as a function of time for the experiment illustrated in FIG. 24. As seen in FIG. 25 the micro diaphragm pump delivered linear performance throughout the experiment. FIG. 26 illustrates accumulated volume as a function of time during the last 300 seconds of the experiment. FIG. 26 suggests that the micro diaphragm pump delivered consistent shot size throughout the test. FIG. 27 is a trumpet curve for the last 100 data points of FIG. 24. The target shot size is set to the average shot size, resulting in zero average error. The maximum spread in shot size is ±2%, which is exceptionally low. This experiment demonstrates that micro diaphragm pumps of the present invention can accurately and precisely draw infusion liquid from the outlet of a pre-filled insulin cartridge, at small shot sizes.

FIG. 28 illustrates outlet pressure as a function of time for an automatically controlled, active, micro diaphragm pump that is connected at its inlet to a pre-filled insulin cartridge. A stroke height of 500 microns and a cycle time of 15 seconds were used. Outlet pressure, as mV output, was measured at the outlet of the micro diaphragm pump. Within three cycles, the outlet pressure reached 90 psi. The experiment was then terminated, due to the limitations of the pressure sensor. It is expected that the micro diaphragm pump can reach much higher pressures. Micro diaphragm pumps quickly reach high pressures because they have low compliance, and their valves seal very well. By comparison, syringe barrels and pistons, as used in syringe pumps, have considerable compliance. In other words, they expand and contract as pressure increases and decreases. The ability of micro diaphragm pumps to generate high pressures within a few cycles is very useful in clearing and detecting occlusions.

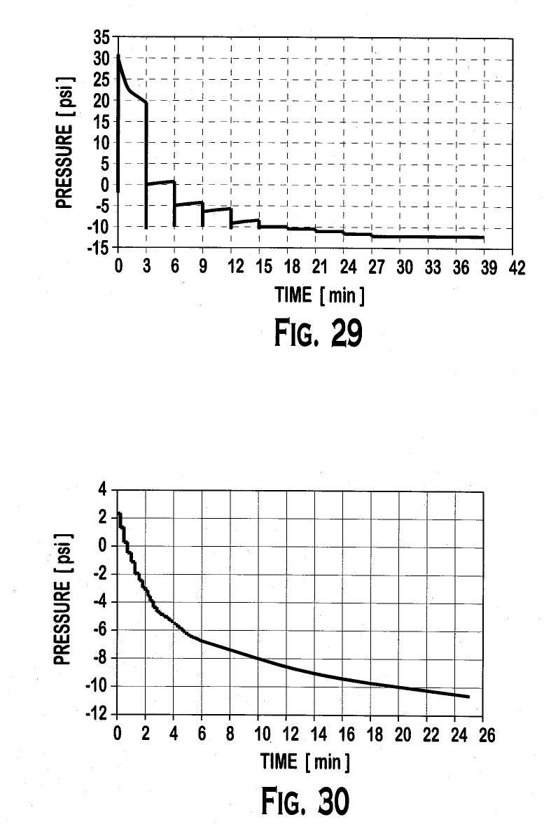

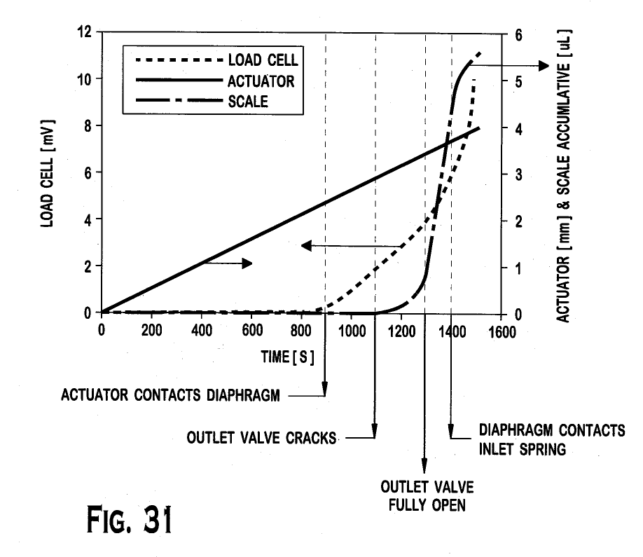

FIG. 29 illustrates inlet pressure as a function of time for an automatically controlled, active, micro diaphragm pump that is connected at its inlet to a vacuum/pressure gauge. A stroke height of 500 microns and a cycle time of 3 minutes were used. Within 8 cycles, an inlet pressure of −12 psi was reached. Between cycles, the inlet and outlet check valves maintained negative pressure and did not leak. Micro diaphragm pumps of the present invention can draw infusion liquid from a pre-filled insulin cartridge because they can generate substantial negative pressure at their inlets. FIG. 30 illustrates inlet pressure as a function of time for an automatically controlled, active, micro diaphragm pump that is connected at its inlet to a vacuum/pressure gauge. In this experiment, a stroke height of 500 microns and a cycle time of 15 seconds were used. Within 24 minutes an inlet pressure of −11 psi was reached.

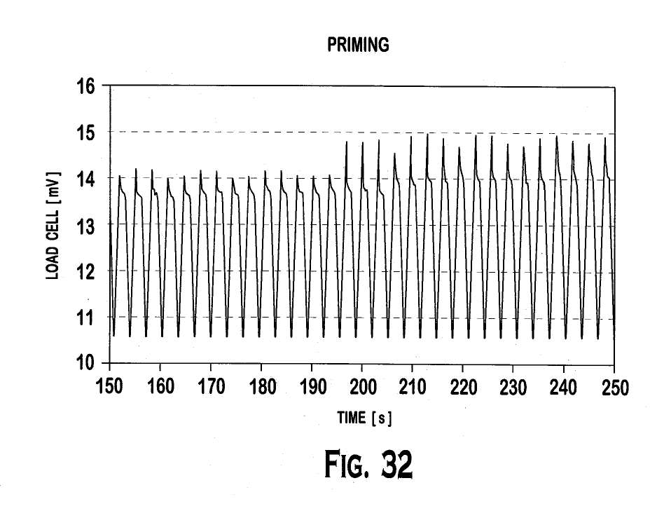

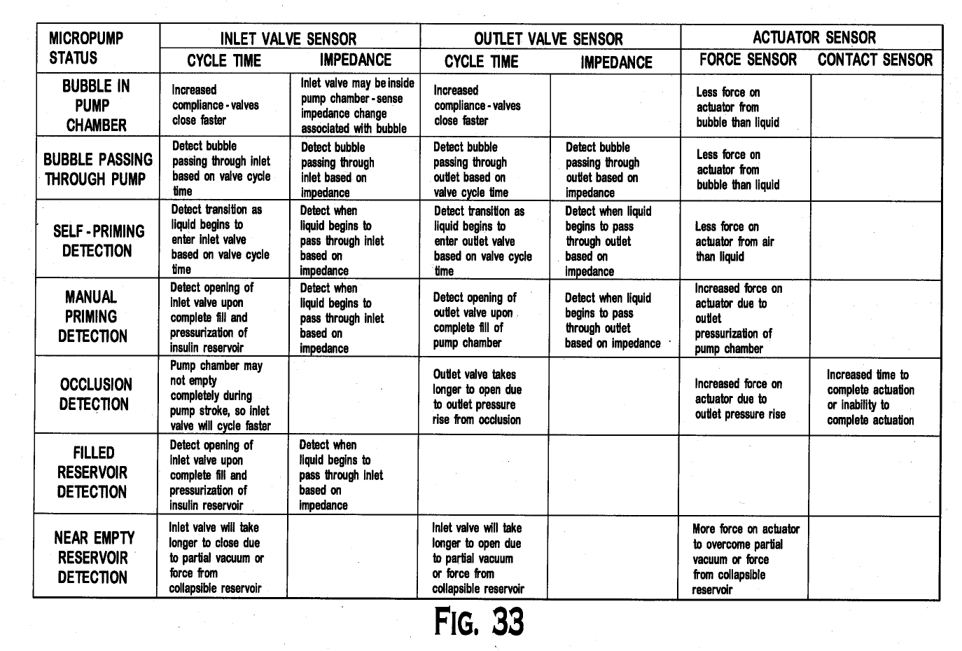

As mentioned previously, and illustrated in FIGS. 2A-2D, sensor 322 can be used to measure forces associated with operation of micro diaphragm pump 300. Sensor 322 is useful in operating micro diaphragm pump 300. For example, if sensor 322 can measure force, it can be used to determine when actuator 316 contacts diaphragm 302, and when diaphragm 302 reaches substrate 304. Sensor 322 can be used to sense when liquid enters the pump chamber, to sense when an empty reservoir introduces air into the pump chamber, or to sense when bubbles enter the pump chamber. FIG. 31 illustrates actuator position (mm), actuator force (mV), and cumulative dispensed volume (microliters) as a function of time during a down stroke, for an automatically controlled, active, micro diaphragm pump that is connected to a force and displacement sensor. A stroke height of 500 microns and a cycle time of 1500 seconds were used. In FIG. 31, actuator force (mV) increases dramatically as the actuator contacts the diaphragm, decreases slightly as the outlet valve cracks (begins to open), decreases slightly as the outlet valve fully opens, and increases sharply as the diaphragm contacts the inlet spring. Cumulative dispensed volume begins when the outlet valve cracks, increases sharply as the outlet valve fully opens, and begins to taper off as the diaphragm contacts the inlet spring. FIG. 31 illustrates that sensors can be used to detect pump status. FIG. 32 illustrates actuator force (mV) as a function of time, for an automatically controlled, active, micro diaphragm pump that is being primed. A stroke height of 500 microns and a cycle time of 3 seconds were used. In FIG. 32, actuator force (mV) increases dramatically as the actuator contacts the diaphragm and inlet spring, as illustrated in the first 14 pump cycles. During the first 14 pump cycles the pump is moving air through its inlet channels and pump chamber. After 14 pump cycles, the pump begins to move infusion liquid, and the magnitude of actuator force increases. The difference in actuator force can be used to detect air and/or liquid in the pump chamber.

As mentioned previously, a variety of sensors can be used in embodiments of the present invention. Force sensors can be used to measure actuator force, displacement sensors can be used to measure actuator position, and electronic sensors can be used to measure the position of the diaphragm, the inlet check valve, and the outlet check valve. Using sensors to measure pump status improves performance in a number of ways. To improve accuracy, sensors can be used to control and verify delivery volumes. As described in the preceding experiment, sensors can be used to detect the presence of air or liquid in the pump chambers and valves. This is useful in detecting bubbles and leaks, as well as the status of priming. During priming, it is useful to know when liquid dispense begins, so as to avoid over or under dosage. Sensors can also be used to detect blockage in infusion lines and cannulas. When blockage occurs, actuator force changes, and check valves may not open or close properly. Sensors can detect when infusion liquid reservoirs have emptied, and when they are full and still delivering infusion liquid. In systems where reservoirs and the pump are filled and primed manually, sensors can be used to alert the user as to the status of the procedure. Force sensors can detect the presence of liquid and air in the pump chamber, while electronic sensors can determine the status of the inlet and outlet valves. An array of actuator and valve sensors can periodically assess the system status, assuring the user that various pump components are functioning properly.

As mentioned previously, pump status can be ascertained if the status of the check valves is known. For example, if a particle is lodged in one or both of the check valves, unwanted forward or backward flow may occur. On the other hand, if a check valve is stuck in the closed position, flow might be blocked. Partial or total occlusion on the outlet side of the pump can prevent the outlet valve from opening, or reduce the amount that it opens. Excessive pressurization of the inlet reservoir can cause both valves to open, and could result in unwanted infusion liquid delivery. When pockets of air or bubbles pass through the pump, less force may be required to open and close inlet and outlet valves, potentially causing malfunctions. If there is a leak in the pump, inlet and outlet valves may not open or close completely, depending on the location of the leak. Siphoning between the inlet and the outlet, or visa versa, may cause the inlet or outlet valve to open when they should be closed.

In embodiments of the present invention, electrically conductive layers or coatings can be incorporated into the inlet and/or outlet valves. Using the conductive layers or coatings, electrical impedance-based measurements can signal when the valves are open, closed, or partially closed. In some embodiments of the present invention, valve springs and disks can include flex circuit material, such as polyimide embedded with conductive layers. Alternatively, valve springs and/or disks can be constructed of a conductive material, such as a conductive polymer or etched thin metal sheet. Optionally, a non-conductive insulating layer can cover portions of the conductive material. Electrical leads to the valve springs and/or disks can be routed to the edge of the device using the flex circuit or conductive material, and can be connected to sensing circuits located in an external or internal controller. When the valve disk contacts the valve seat plate, an electrical connection can be made, signaling that the valve is closed. Similarly, when the valve disk moves off of the valve seat plate, the electrical contact can be broken, signaling that the valve is open. The amount of force or time that it takes for a valve to open and close may indicate whether air or liquid is passing through the pump, allowing for the detection of bubbles and priming. When a valve is open, the impedance between the valve disk and valve seat plate will vary, depending on whether air or liquid is in the pump. This provides another method for bubble and priming detection. The ability to monitor both valves provides more information regarding the status of the pump than using information based only on the diaphragm or actuator. For example, using valve sensors allow the system to determine if the inlet valve or outlet valve is stuck open or closed. By sensing at both valves, it is possible to monitor air bubbles as they first pass through the inlet valve, then pass through the outlet valve. It is also possible to determine if a bubble moves into the pump chamber through the inlet valve, but does not exit.

In some embodiments of the present invention, pump status is determined using measurements related to the actuator. Force sensors, contact sensors, or position sensors can be coupled with the actuator to confirm proper operation. If the actuator does not behave appropriately, sensors can detect the problem and alert the user. Sensors can verify proper motion of the actuator, can detect bubbles in the pump chamber (reduced force on actuator), and can detect occlusions (increased force on actuator). Simple electrical contacts on the surface of the diaphragm can create an electrical switch when contact is made between the diaphragm and the actuator, verifying motion of the actuator, as well as alignment between the actuator and diaphragm. As mentioned previously, force on the actuator will be different if there is air or liquid in the pump chamber. During the down stroke, the amount of time it takes for the actuator to reach the inlet spring will vary if there is air or liquid in the pump chamber. The force and time required for the actuator to move up and down will vary if the inlet and/or outlet valves are stuck open or closed. The force and time required for the actuator to move up and down will vary depending upon backpressure at the pump's outlet side. The force and time required for the actuator to move up and down will vary depending upon pressure in the pump's reservoir. The force and time required for the actuator to move up and down will vary if there is an occlusion at the pump's inlet or outlet. Alignment of the actuator and the diaphragm can be determined based on force at the actuator. Alignment of the actuator and the diaphragm can also be determined using electrical contact between the actuator and the diaphragm. As mentioned previously, a sharp rise in force at the actuator occurs when the diaphragm contacts the inlet spring and/or the valve plate seat.

Embodiments of the present invention can be used to deliver drugs, cells, DNA, biopharmaceuticals, and conventional pharmaceuticals, in the treatment of various disorders, including Parkinson's disease, epilepsy, pain, immune system diseases, inflammatory diseases, obesity, and diabetes. Embodiments of the present invention can also be used to deliver GLP-1 drugs, such as Symlin, Byetta, etc.

Although embodiments of the present invention have been described in respect to a micro diaphragm pump, elements of the present invention can be incorporated into piston based micro pumps. In those embodiments, the diaphragm is replaced by a moving bellows, or by a piston with a sliding seal (such as an o-ring).

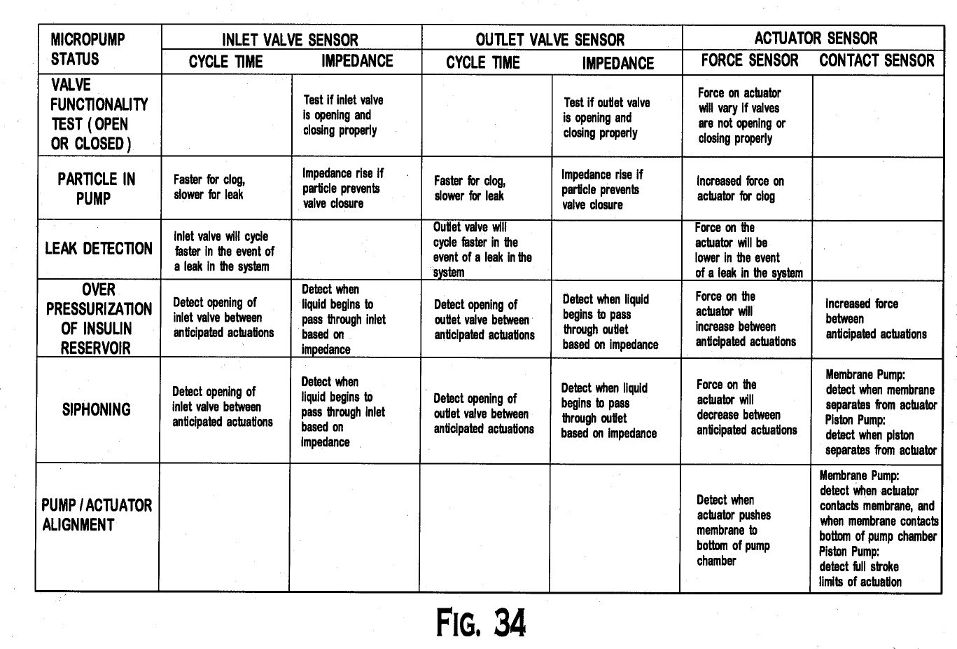

FIGS. 33 and 34 illustrate various micro diaphragm pump status conditions that can be ascertained using inlet valve sensors, outlet valve sensors, and actuator sensors, according to embodiments of the present invention. As mentioned previously, inlet and outlet valve sensors can include measurements of cycle time (via electrical contact sensors), and measurements of electrical impedance. Actuator sensors can include measurements of force required to move the actuator, along with electrical contacts between the actuator, diaphragm, and other pump components. FIGS. 33 and 34 include detailed description of the micro pump status and the state of the inlet valve sensors, the outlet valve sensors, and the actuator sensors. The state of the inlet valve sensors, outlet valve sensors, and the actuator sensors can be used individually, or coupled, in determining the status of the micro pump.

What is claimed is:

1. A micro diaphragm pump for delivering infusion liquid comprising:
a pump chamber;
a diaphragm, that is connected to and partially defines the border of said pump chamber;
an inlet channel with inlet channel proximal end and inlet channel distal end, connected at said inlet channel distal end to said pump chamber;
an outlet channel with outlet channel proximal end and outlet channel distal end, connected at said outlet channel proximal end to said pump chamber;
an inlet check valve with inlet spring and inlet disk, located between said inlet channel distal end and said pump chamber;
an outlet check valve with outlet spring and outlet disk, located between said pump chamber and said outlet channel proximal end; and,
an actuator, which is in intermittent contact with said diaphragm.

2. A micro diaphragm pump for delivering infusion liquid as claimed in claim 1 further comprising a sensor which is in proximity to said actuator.

3. A micro diaphragm pump for delivering infusion liquid as claimed in claim 1 further comprising a sensor which is in proximity to said diaphragm.

4. A micro diaphragm pump for delivering infusion liquid as claimed in claim 1 further comprising a sensor which is in proximity to said inlet check valve.

5. A micro diaphragm pump for delivering infusion liquid as claimed in claim 1 further comprising a sensor which is in proximity to said outlet check valve.

6. A micro diaphragm pump for delivering infusion liquid as claimed in claim 1 further comprising an over-pressure check valve connected between said inlet channel proximal end and said inlet channel distal end.

7. A micro diaphragm pump for delivering infusion liquid as claimed in claim 1 wherein said inlet channel is connected to a reservoir at said inlet channel proximal end.

8. A micro diaphragm pump for delivering infusion liquid as claimed in claim 7 wherein said reservoir is a syringe reservoir.

9. A micro diaphragm pump for delivering infusion liquid as claimed in claim 7 wherein said reservoir is a collapsible reservoir.

10. A micro diaphragm pump for delivering infusion liquid as claimed in claim 1 wherein said outlet channel is connected to an infusion line at said outlet channel distal end.

11. A micro diaphragm pump for delivering infusion liquid as claimed in claim 10 wherein said infusion line is connected to a cannula.

12. A micro diaphragm pump for delivering infusion liquid as claimed in claim 1 wherein said inlet disk is made of natural rubber.

13. A micro diaphragm pump for delivering infusion liquid as claimed in claim 1 wherein said inlet disk is made of an elastomer.

14. A micro diaphragm pump for delivering infusion liquid as claimed in claim 1 wherein said outlet disk is made of natural rubber.

15. A micro diaphragm pump for delivering infusion liquid as claimed in claim 1 wherein said outlet disk is made of an elastomer.

16. A micro diaphragm pump for delivering infusion liquid as claimed in claim 1 wherein said inlet disk is thinner than said outlet disk.

17. A micro diaphragm pump for delivering infusion liquid as claimed in claim 1 wherein said inlet spring and inlet disk self-align to said inlet channel distal end.

18. A micro diaphragm pump for delivering infusion liquid as claimed in claim 1 wherein said outlet spring and outlet disk self-align to said outlet channel proximal end.

19. A micro diaphragm pump for delivering infusion liquid as claimed in claim 1 wherein said inlet disk is larger in diameter than said inlet channel distal end.

20. A micro diaphragm pump for delivering infusion liquid as claimed in claim 1 wherein said outlet disk is larger in diameter than said outlet channel proximal end.

21. A micro diaphragm pump for delivering infusion liquid as claimed in claim 1 wherein said inlet spring is stretched away from said inlet channel distal end by said inlet disk.

22. A micro diaphragm pump for delivering infusion liquid as claimed in claim 1 wherein said outlet spring is stretched away from said outlet channel proximal end by said outlet disk.

23. A micro diaphragm pump for delivering infusion liquid as claimed in claim 1 wherein said inlet spring is attached to said inlet disk.

24. A micro diaphragm pump for delivering infusion liquid as claimed in claim 1 wherein said outlet spring is attached to said outlet disk.

25. A micro diaphragm pump for delivering infusion liquid as claimed in claim 1 wherein said inlet check valve has a lower opening pressure than said outlet check valve.

26. A micro diaphragm pump for delivering infusion liquid as claimed in claim 1 wherein said outlet check valve has a lower opening pressure than said inlet check valve.

27. A micro diaphragm pump for delivering infusion liquid as claimed in claim 1 wherein said inlet check valve and said outlet check valve have the same opening pressure.

28. A micro diaphragm pump for delivering infusion liquid as claimed in claim 1 wherein said diaphragm conforms to said pump chamber when displaced by said actuator.

29. A micro diaphragm pump for delivering infusion liquid as claimed in claim 1 wherein said inlet spring is flat and spiral shaped.

30. A micro diaphragm pump for delivering infusion liquid as claimed in claim 1 wherein said outlet spring is flat and spiral shaped.

31. A micro diaphragm pump for delivering infusion liquid as claimed in claim 1 wherein said outlet spring is thicker than said inlet spring.

32. A micro diaphragm pump for delivering infusion liquid as claimed in claim 1 wherein said outlet spring has a higher force constant than said inlet spring.

33. A method of delivering infusion liquid comprising the steps of:
drawing infusion liquid into a pump chamber by moving an actuator and diaphragm into a first position; and,
expelling infusion liquid from said pump chamber by moving said actuator and said diaphragm into a second position;
wherein said infusion liquid flows through an inlet channel and an inlet check valve with inlet spring and inlet disk while being drawn into said pump chamber, and said infusion liquid flows through an outlet channel and an outlet check valve with outlet spring and outlet disk while being expelled from said pump chamber.

34. A method of delivering infusion liquid as claimed in claim 33 wherein the position of said actuator is determined by a sensor.

35. A method of delivering infusion liquid as claimed in claim 33 wherein the position of said diaphragm is determined by a sensor.

36. A method of delivering infusion liquid as claimed in claim 33 wherein the position of said inlet check valve is determined by a sensor.

37. A method of delivering infusion liquid as claimed in claim 33 wherein the position of said outlet check valve is determined by a sensor.

38. A method of delivering infusion liquid as claimed in claim 33 wherein said infusion liquid flows through an over-pressure check valve while being drawn into said pump chamber.

39. A method of delivering infusion liquid as claimed in claim 33 wherein said inlet channel is connected to a reservoir and said infusion liquid is drawn from said reservoir into said pump chamber.

40. A method of delivering infusion liquid as claimed in claim 33 wherein said inlet channel is connected to a syringe reservoir and said infusion liquid is drawn from said syringe reservoir into said pump chamber.

41. A method of delivering infusion liquid as claimed in claim 33 wherein said inlet channel is connected to a collapsible reservoir and said infusion liquid is drawn from said collapsible reservoir into said pump chamber.

42. A method of delivering infusion liquid as claimed in claim 33 wherein said outlet channel is connected to an infusion line.

43. A method of delivering infusion liquid as claimed in claim 42 wherein said infusion line is connected to a cannula.

44. A method of delivering infusion liquid as claimed in claim 33 wherein said inlet disk is made of natural rubber.

45. A method of delivering infusion liquid as claimed in claim 42 wherein said inlet disk is made of an elastomer.

46. A method of delivering infusion liquid as claimed in claim 33 wherein said outlet disk is made of natural rubber.

47. A method of delivering infusion liquid as claimed in claim 33 wherein said outlet disk is made of an elastomer.

48. A method of delivering infusion liquid as claimed in claim 33 wherein said inlet disk is thinner than said outlet disk.

49. A method of delivering infusion liquid as claimed in claim 33 wherein said inlet spring and inlet disk self-align to said inlet channel.

50. A method of delivering infusion liquid as claimed in claim 33 wherein said outlet spring and outlet disk self-align to said outlet channel.

51. A method of delivering infusion liquid as claimed in claim 33 wherein said inlet disk is larger in diameter than said inlet channel.

52. A method of delivering infusion liquid as claimed in claim 33 wherein said outlet disk is larger in diameter than said outlet channel.

53. A method of delivering infusion liquid as claimed in claim 33 wherein said inlet spring is stretched by said inlet disk.

54. A method of delivering infusion liquid as claimed in claim 33 wherein said outlet spring is stretched by said outlet disk.

55. A method of delivering infusion liquid as claimed in claim 33 wherein said inlet spring is attached to said inlet disk.

56. A method of delivering infusion liquid as claimed in claim 33 wherein said outlet spring is attached to said outlet disk.

57. A method of delivering infusion liquid as claimed in claim 33 wherein said inlet check valve has a lower opening pressure than said outlet check valve.

58. A method of delivering infusion liquid as claimed in claim 33 wherein said outlet check valve has a lower opening pressure than said inlet check valve.

59. A method of delivering infusion liquid as claimed in claim 33 wherein said inlet check valve and said outlet check valve have the same opening pressure.

60. A method of delivering infusion liquid as claimed in claim 33 wherein said diaphragm conforms to said pump chamber when said actuator and said diaphragm are moved to said second position.

61. A method of delivering infusion liquid as claimed in claim 33 wherein said inlet spring is flat and spiral shaped.

62. A method of delivering infusion liquid as claimed in claim 33 wherein said outlet spring is flat and spiral shaped.

63. A method of delivering infusion liquid as claimed in claim 33 wherein said outlet spring is thicker than said inlet spring.

64. A method of delivering infusion liquid as claimed in claim 33 wherein said outlet spring has a higher force constant than said inlet spring.
